(19) United States
(12) Patent Application Publication
(10) Pub. No.: US 2018/0344535 A1
(43) Pub. Date: Dec. 6, 2018

(54) TAMPAD FEMININE MENSTRUAL PAD

(71) Applicant: Amber Adams, Intervale, NH (US)

(72) Inventor: Amber Adams, Intervale, NH (US)

(21) Appl. No.: 15/614,263

(22) Filed: Jun. 5, 2017

(51) Int. Cl. A61F 13/20; A61F 13/472; A61F 13/505

(52) U.S. Cl. CPC A61F 13/2051; A61F 2013/4729; A61F 13/505; A61F 13/47227

(57) ABSTRACT

An absorbent menstrual pad with an integrated conically shaped vaginal insert is provided. In some embodiments, the present invention may provide for an integrated conically shaped vaginal insert that has a base ridge consisting of a higher absorbent material. The present invention may also provide perforated connections allowing a user to modify the width and length of the absorbent menstrual pad and remove the wings from the menstrual pad.

### CROSS-REFERENCE TO RELATED APPLICATION

Not applicable.

### TECHNICAL FIELD

The present invention relates generally to the field of tampons and menstrual pads and, more specifically, to the combination of an absorbent vaginal insert and a menstrual pad.

### BACKGROUND

Many women find tampons more convenient and comfortable to wear than menstrual pads. However, mensural pads are often used in conjunction with tampons due to the increased risk that the menstrual blood cannot be fully absorbed by the tampon and may leak when the tampon is full or when there is clearance between the vaginal wall and the tampon. Wearing a tampon insert along with a menstrual pad protects against the risk of leaked menstrual blood and prevents against the unnecessary staining of a woman's underwear or clothing. Notwithstanding the presence of a tampon insert, even minimal amounts of menstrual blood leaking from the vaginal cavity can overflow a small menstrual pad. Additionally, many menstrual pads of sufficient absorbent strength may still result in a menstrual blood overflow onto a woman's clothing when a woman moves a certain way or lays in a certain direction. This may usually be corrected by using a “winged” menstrual pad, however, not all women find winged menstrual pads to be comfortable or useful. Although many women have the option of changing their tampon frequently during the day, it is difficult to ascertain when a woman's increased heartrate, sudden body movement, or the mere passage of time will impact a woman's menstrual blood flow, resulting in unwanted leakage. Women who suffer from menstrual blood overflow would benefit from using a menstrual pad along with a tampon. As such, a need exists for a menstrual pad that has an integrated absorbent vaginal insert with removable wings.

Various attempts to solve this problem have been made. One such attempt can be seen with respect to U.S. Pat. No. 6,059,763, incorporated by reference in its entirety herein, which generally discloses an absorbent menstrual pad with a removable tampon located in the central portion of the menstrual pad. While this disclosure does provide for a menstrual pad with an integrated tampon, it fails to provide for a menstrual pad with an integrated absorbent vaginal insert that has sufficient absorbency, and is also not configured to allow for removable menstrual pad wings.

Another attempt can be seen with respect to U.S. Pat. No. 6,863,664 B2, incorporated by reference in its entirety herein, which generally discloses an absorbent pad tailored for use with a tampon. While this disclosure does provide for a menstrual pad with an integrated tampon, it fails to provide for a menstrual pad with an integrated absorbent vaginal insert where the menstrual pad has sufficient absorbency. This disclosure would also be very difficult to insert into the vaginal cavity.

Yet another attempt can be seen with respect to U.S. Pat. No. 6,447,495 B1, incorporated by reference in its entirety herein, which generally discloses a winged menstrual pad with sufficient means to secure the menstrual pad and the accompanying wings to the woman's undergarments. While this disclosure does provide for a winged menstrual pad, it fails to disclose a menstrual pad with wings that may be removed.

Various attempts have been made to solve the problems which may be found in the related art but have thus far been unsuccessful. A need exists for a new menstrual pad with an integrated absorbent vaginal insert that has removable wings that may secure to the woman's undergarments.

### SUMMARY OF THE INVENTION

It is to be understood that in the present disclosure, all embodiments are provided as illustrative and non-limiting representatives of many possible embodiments. In addition, the terms “is,” “can,” “will,” and the like are herein used as synonyms for and interchangeable with terms such as “may,” “may provide for,” and “it is contemplated that the present invention may” and so forth.

The present invention relates to a menstrual pad configured with an integrated absorbent vaginal insert that overcomes the deficiencies of the prior art. It is contemplated that the present invention may provide easily removable menstrual pad wings.

For purposes of summarizing, certain aspects, advantages, and novel features of the present invention are provided herein. It is to be understood that not all such aspects, advantages, or novel features may be provided in any one particular embodiment. Thus, the disclosed subject matter may be embodied or carried out in a manner that achieves or optimizes one aspect, advantage, or novel feature or group of features without achieving all aspects, advantages, or novel features as may be taught or suggested.

In view of the foregoing disadvantages inherent in the known art, the present invention provides a menstrual pad with an integrated absorbent vaginal insert. The general purpose of the present invention, which will be described subsequently in greater detail, is to provide a menstrual pad with an integrated absorbent vaginal insert that is generally easy to use and modify based upon the user's preferences. The features of the invention which are believed to be novel are particularly pointed out and distinctly claimed in the concluding portion of the specification. These and other features, aspects, and advantages of the present invention will become better understood with reference to the following drawings and detailed description.

In one embodiment, the present invention may provide for a conically shaped vaginal insert having absorbent properties configured to rest on the top layer of the menstrual pad. The conical shaped vaginal insert is designed to absorb menstrual blood that flows from the vaginal cavity. The present invention may also provide a ridge at the base of the conically shaped vaginal insert with a greater level of absorbency to prevent against unnecessary spillage from the conically shaped vaginal insert to the menstrual pad.

In another embodiment, the present invention may provide for perforated edges to allow a user to adjust the size of the menstrual pad at the user's discretion. The menstrual pad may be designed with a perforated edge round the perimeter of the menstrual pad allowing at least a ¼ inch clearance from the outer edge of the menstrual pad. The present invention may also have a secondary perforated edge extending along right and left side of the menstrual pad having at least a ⅛ inch clearance on the outer body of the primary perforated edge. The secondary perforated edge is designed to have “wings” extending along the outer body of the secondary perforated edge. The wings of the present invention can detach from the menstrual pad at the secondary perforated edge at the user's discretion.

To use the menstrual pad, a user may simply remove the protective separators from the adhesive zones located on the bottom portion of the menstrual pad and on the first and second wings located on the right and left sides of the menstrual pad. Once removed, the user may secure the menstrual pad to the vaginal/crotch area located on the inside of a woman's undergarments by pressing the center adhesive zone of the menstrual pad firmly to the undergarment. If present, the first and second wings may be secured to the undergarment by folding the wings under the outside layer of undergarment's vaginal/crotch area, pressing firmly to hold the wings in place. Once in place, the user would simply pull up the undergarments and place the conically shaped vaginal insert in the inner folds of the vaginal area.

To remove the menstrual pad, the user would simply lift the menstrual pad from the vaginal/crotch area of the undergarment. If present, the wings may be removed by peeling the adhesive zone away from the undergarment. To modify the size of the menstrual pad or to remove the first and second wings, the user may hold the center area of the menstrual pad while simultaneously pulling firmly on the perforated edges.

The unique features of the present invention may provide the following benefits for one or more consumers: it may be cleaner than using a regular menstrual pad along; young girls may find it easier to use when starting their periods; it may be less noticeable than a normal menstrual pad.

### BRIEF DESCRIPTION OF THE DRAWINGS

These and other features, aspects and advantages of the present invention will become better understood with regard to the following description, appended claims, and accompanying figures where:

FIG. 1 shows a perspective view of a menstrual pad in accordance with at least one embodiment of the invention.

FIG. 2 shows a top view of a menstrual pad accordance with at least one embodiment of the invention.

FIG. 3 shows a bottom view a menstrual pad in accordance with at least one embodiment of the invention.

FIG. 4 shows a right side view of a menstrual pad in accordance with one embodiment of the invention.

FIG. 5 shows a left side view of a menstrual pad in accordance with one embodiment of the invention.

FIG. 6 shows a front view of a menstrual pad in accordance with one embodiment of the invention.

FIG. 7 shows a bottom view of a menstrual pad in accordance with one embodiment of the invention.

### DETAILED DESCRIPTION

The present invention overcomes the limitations of the prior art by providing a new and more effective menstrual pad with an integrated absorbent vaginal insert.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any embodiment or element of an embodiment disclosed in this disclosure will be determined by its intended use.

It is to be understood that the drawings and the associated descriptions are provided to illustrate potential embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment,” “at least one embodiment,” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific detail. Well known features, elements or techniques may not be shown in detail in order not to obscure the embodiments.

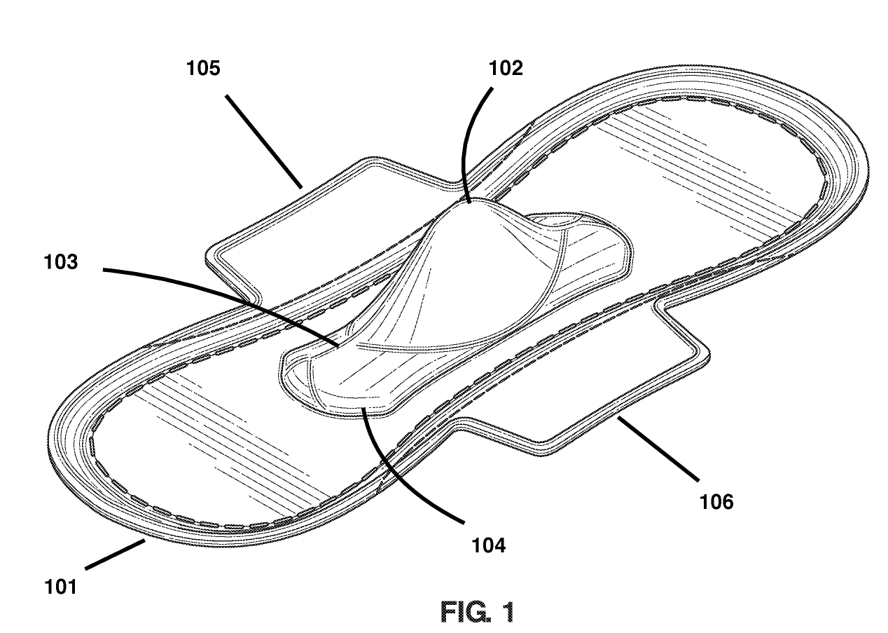

Turning attention to FIG. 1, a top perspective view of a menstrual pad in accordance with at least one embodiment of the invention is shown. In the embodiment depicted, a viewer may perceive a menstrual pad 101, with an integrated conically shaped vaginal insert 102, having a base member that is parallel to the top layer of the menstrual pad 103, with a ridge having additional absorbency strength 104. In some embodiments, the present invention may provide for wings on the on the right side 105 and left side 106 of the menstrual pad 101.

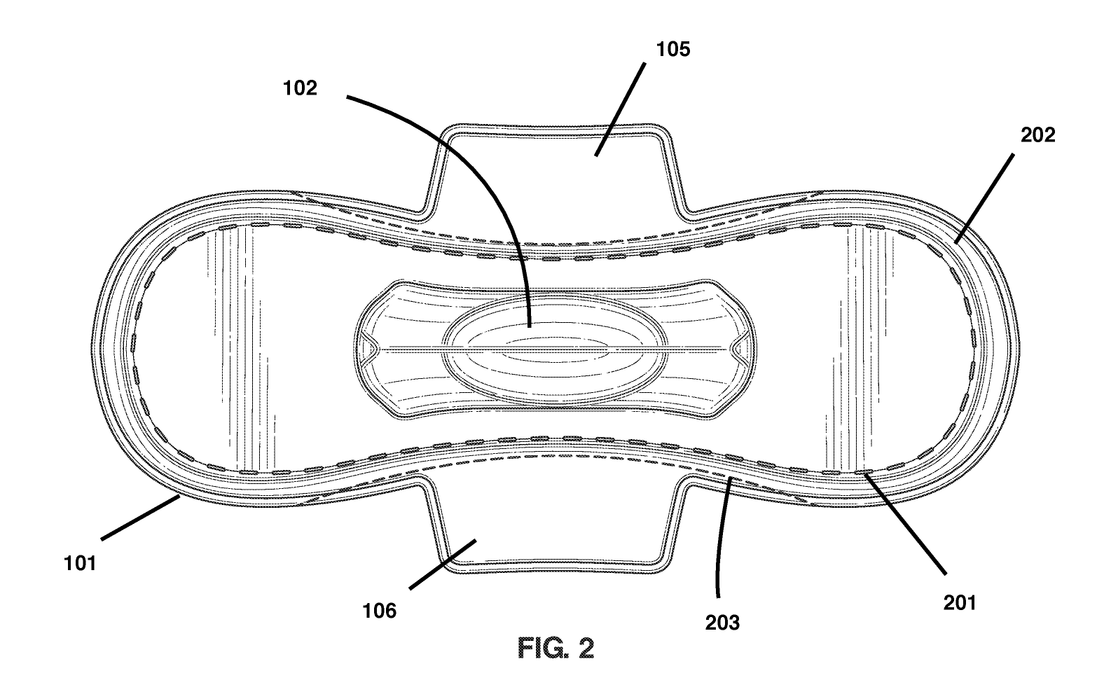

With respect to FIG. 2, a top view of a menstrual pad accordance with at least one embodiment of the invention is shown. In the embodiment depicted, a viewer may perceive a perforated line 201 that is at least ¼ inch from the menstrual pad edge 202, with a secondary perforated line 203 that is at least ⅛ inch from the outer edge of the primary perforated line 201 and the inner edge of the secondary perforated line 203 allowing the right wing 105 and left wing 106 to separate from the menstrual pad 101.

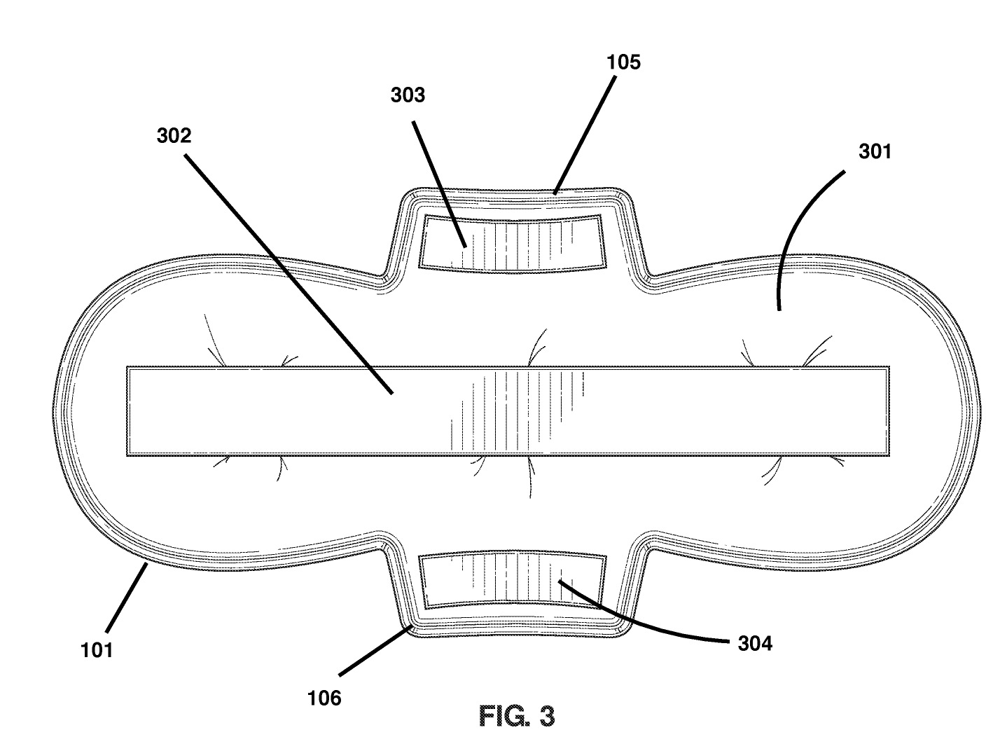

Turning attention to FIG. 3, a bottom view a menstrual pad in accordance with at least one embodiment of the invention is shown. In the embodiment depicted, a viewer may perceive the bottom 301 of a menstrual pad 101 having a central adhesive zone 302 that extends parallel along the length of the menstrual pad 101. In some embodiments, the present invention may include an adhesive zone 303 on the bottom of the right wing 105 and an adhesive zone 304 on the bottom of the left wing 106.

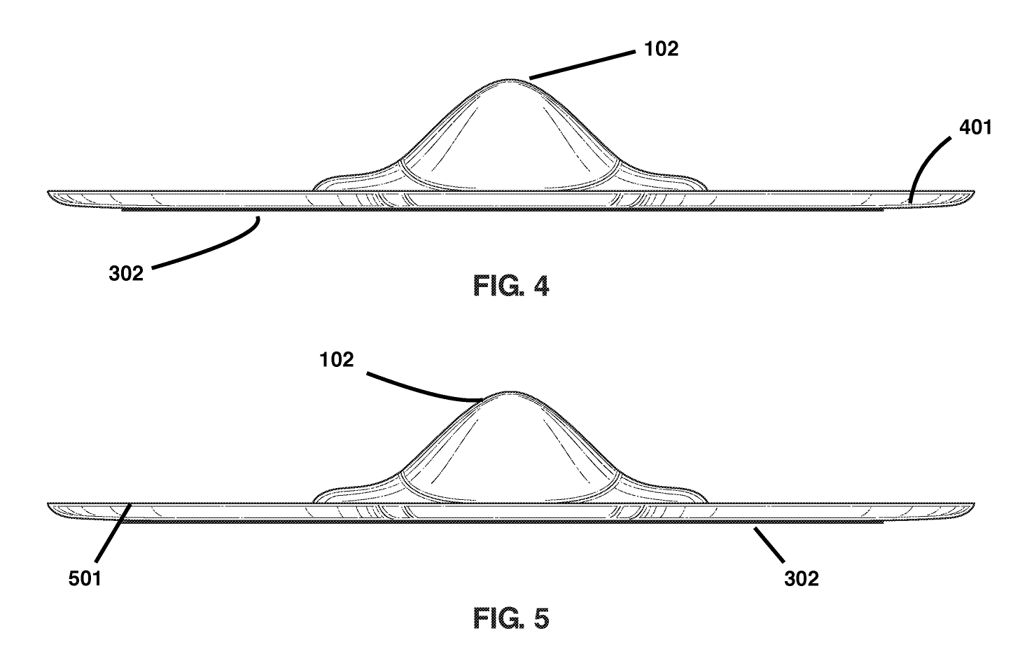

With respect to FIG. 4, a right side view of a menstrual pad in accordance with one embodiment of the invention is shown. In the embodiment depicted, a viewer may perceive the relative thickness 401 of the menstrual pad 101 in relation to the size and shape of the integrated conically shaped vaginal insert 102 and the centrally located adhesive zone 302.

With respect to FIG. 5, a left side view of a menstrual pad in accordance with one embodiment of the invention is shown. In the embodiment depicted, a viewer may perceive the relative thickness 501 of the menstrual pad 101 in relation to the size and shape of the integrated conically shaped vaginal insert 102 and the centrally located adhesive zone 302.

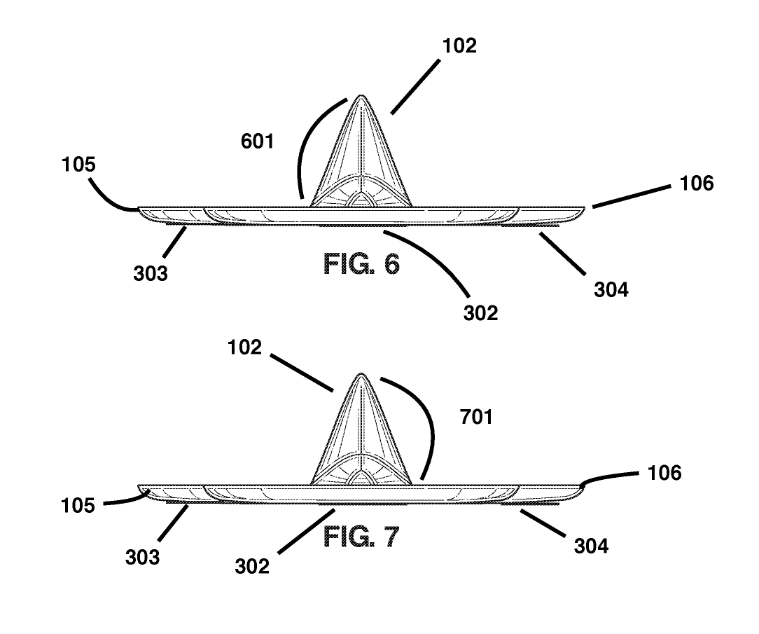

Turning attention to FIG. 6, a front view of a menstrual pad in accordance with one embodiment of the invention is shown. In the embodiment depicted, a viewer may perceive the width of the right wing 105 and the left wing 106 in relation to the height 601 of the integrated conically shaped vaginal insert 102.

With respect to FIG. 7, a rear view of a menstrual pad in accordance with one embodiment of the invention is shown. In the embodiment depicted, a viewer may perceive the width of the right wing 105 and the left wing 106 in relation to the height 701 of the integrated conically shaped vaginal insert 102.

### Conclusions, Ramifications, and Scope

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

While the menstrual pad configured with an integrated conically shaped vaginal insert with removably attached right and left wings generally described herein has been disclosed in connection with a number of embodiments shown and described in detail, various modifications should be readily apparent to those of skill in the art.

What is claimed is:

1. An absorbent menstrual pad for absorbing menstrual blood that is expelled from a user's vaginal cavity, comprising:
a base menstrual pad with first and second length sides, and;
an integrated conically shaped vaginal insert comprised of fluid absorbing material extending substantially perpendicular to the base menstrual pad, located at a central position on the menstrual pad from the menstrual pad's front, rear, right, and left edges.

2. An absorbent menstrual pad of claim 1, wherein the integrated conically shaped vaginal insert is made of high absorbent material.

3. An absorbent menstrual pad as recite in claim 2, wherein the integrated conically shaped vaginal insert has a base ridge, having a higher level of absorbency than the conically shaped vaginal insert, and connecting to the top layer of the base menstrual pad.

4. An absorbent menstrual pad of claim 1, wherein the absorbent menstrual pad consists of a core fluid absorbing material positioned between top absorbent layer and a bottom non-absorbent layer.

5. An absorbent menstrual pad as recited in claim 4, wherein the bottom non-absorbent layer has a centrally positioned adhesive zone extending parallel along the length of the menstrual pad, where said centrally positioned adhesive zone has a peelably applied separating material that can be peeled off of the centrally positioned adhesive zone allowing the menstrual pad to secure to an undergarment surface.

6. An absorbent menstrual pad for absorbing menstrual blood that is expelled from a user's vaginal cavity, comprising:
a base menstrual pad with first and second length sides;
an integrated conically shaped vaginal insert comprised of fluid absorbing material extending substantially perpendicular to the base menstrual pad, located at a central position on the menstrual pad from the menstrual pad's front, rear, right, and left edges; and
first and second wings centrally positioned along the first and second length sides of the base menstrual pad and the integrated conically shaped vaginal insert.

7. An absorbent menstrual pad of claim 6, comprising a primary perforated connector extending around the perimeter of the base menstrual pad.

8. An absorbent menstrual pad as recited in claim 7, wherein the primary perforated connector is positioned at least ¼ inch from the menstrual pad's front, rear, right, and left edges.

9. An absorbent menstrual pad of claim 6, comprising a secondary perforated connector positioned on the left and right side of the base menstrual pad.

10. An absorbent menstrual pad as recited in claim 9, wherein the secondary perforated connector is positioned at least ⅛ inch from the outer edge of the primary perforated connector.

11. An absorbent menstrual pad as recited in claim 10, wherein the outer edge of the secondary perforated connector is attached to the first and second wings centrally positioned along the first and second length sides of the base menstrual pad.

12. An absorbent menstrual pad of claim 6, wherein the absorbent menstrual pad consists of a core fluid absorbing material positioned between top absorbent layer and a bottom non-absorbent layer.

13. An absorbent menstrual pad as recited in claim 12, wherein the bottom non-absorbent layer has an adhesive zone extending parallel along the length of the menstrual pad and parallel along the length of the first and second wings, where said adhesive zones have a peelably applied separating material that can be peeled off of the adhesive zones allowing the menstrual pad to secure to an undergarment surface.
